(19) United States
(12) Patent Application Publication
(10) Pub. No.: US 2025/0032598 A1
(43) Pub. Date: Jan. 30, 2025

(54) Compounds, Pharmaceuticals, Formulations, and Methods for Treating and Preventing RNA Virus Infections and Associated Diseases

(71) Applicants: Alexandre Vasilievich Ivachtchenko, Hallandale, FL (US); Alena Alexandrovna Ivachtchenko, Hallandale, FL (US); Nikolay Filippovich Savchuk, Rancho Santa Fe, CA (US); ASAVI LLC, Hallandale, FL (US)

(72) Inventors: Alexandre Vasilievich Ivachtchenko, Hallandale, FL (US); Andrey Alexandrovich Ivashchenko, Moscow (RU); Alena Alexandrovna Ivachtchenko, Hallandale, FL (US); Ilya Andreevich Ivashchenko, Dolgoprudniy, Moscow region (RU); Nikolay Filippovich Savchuk, Rancho Santa Fe, CA (US)

(21) Appl. No.: 18/357,485

(22) Filed: Jul. 24, 2023

(51) Int. Cl. A61K 38/57; A61K 9/127; A61P 31/14; A61P 31/16

(52) U.S. Cl. CPC A61K 38/57; A61K 9/127; A61P 31/14; A61P 31/16

(57) ABSTRACT

The present application is directed to compounds, pharmaceuticals, formulations, and methods for treating and preventing RNA virus infections, particularly infections in mammals. The present application also relates to treating and preventing diseases caused by RNA viruses. In preferred embodiments, the pharmaceuticals, formulations, and methods for treatment and prevention use liposomal aprotinin (Lip-APR).

### FIELD OF THE INVENTION

The present application is directed to compounds, pharmaceuticals, formulations, and methods for treating and preventing RNA virus infections, particularly infections in mammals. The present application also relates to treating and preventing diseases caused by RNA viruses. In preferred embodiments, the pharmaceuticals, formulations, and methods for treatment and prevention use liposomal aprotinin (Lip-APR).

### BACKGROUND OF THE INVENTION

An RNA virus is a virus that has ribonucleic acid (RNA) as its genetic material. The nucleic acid is usually single-stranded RNA (ssRNA) but may be double-stranded (dsRNA). Notable human diseases caused by RNA viruses include the common cold, influenza and acute respiratory diseases (ARD), coronaviruses (SARS-CoV or SARS-CoV-1, MERS-CoV, SARS-CoV-2), Dengue Virus, hepatitis C, hepatitis E, West Nile fever, Ebola virus disease, measles, and others.

Many of the RNA viruses cause severe diseases. For example, influenza can cause mild to severe illness and even death. The complications of influenza [Biggers, A. Flu Complications. Healthline, 2019. https://www.healthline.com/health/flu-complication] could be, for example, pneumonia caused by the influenza virus or bacteria that enter the lungs when the body's defense system is weakened by the flu or dilated cardiomyopathy after influenza A virus infection [Pan, H. Y. et al. Ectopic trypsin in the myocardium promotes dilated cardiomyopathy after influenza A virus infection. Am J Physiol Heart Circ Physiol, 2014, 307, H922-H932. doi: 10.1152/ajpheart.00076.2014].

In studies focusing on the recent COVID-19 pandemic, the highest SARS-CoV-2 copies per cell were detected in the respiratory tract, and lower levels were detected in the kidneys, liver, heart, brain, and blood. These findings indicate a broad organotropism of SARS-CoV-2 [Puelles, V. G. et al. Multiorgan and Renal Tropism of SARS-CoV-2. N Engl J Med. 2020, 383, 590-592. doi: 10.1056/NEJMc2011400.]. It was also observed that at least a third of those infected with SARS-CoV-2 do not develop noticeable symptoms [Oran, D. P.; Topol E. J. The Proportion of SARS-CoV-2 Infections That Are Asymptomatic: A Systematic Review. Annals of Internal Medicine. 2021, 174, 655-662. doi: 10.7326/M20-6976.]. Of those who develop symptoms noticeable enough to be classified as patients, most (81%) develop mild to moderate symptoms (up to mild pneumonia), 14% develop severe symptoms (dyspnea, hypoxia, or more than 50% lung involvement), and 5% develop critical symptoms (respiratory failure, shock, or multiorgan dysfunction) [CDC. Interim Clinical Guidance for Management of Patients with Confirmed Coronavirus Disease (COVID-19). Jun. 30, 2020. https://stacks.cdc.gov/view/cdc/8998].

The anti-RNA drug aprotinin (APR) is used to treat and prevent RNA virus infection in mammals and related diseases caused by RNA viruses.

APR has been actively studied as a preventive and therapeutic agent for treating influenza and acute respiratory diseases (ARD) since the 1980s [Zhirnov, O. P. et al. Suppression of Influenza Virus Replication in Infected Mice by Protease Inhibitors. J. Gen. Virol. 1984, 65, 191-196; doi: 10.1099/0022-1317-65-1-191.].

In 1991, a method was proposed for treating and preventing respiratory ailments of viral or viral-bacterial origin using an acrosol composition containing, as an active ingredient, an inhibitor of proteases selected from aprotinin and derivatives thereof, said active ingredient being dissolved in water to form a solution, said method comprising inhalation or direct application of a therapeutically effective amount of the aerosol on the respiratory tract [Zhirnov, O. P. et al. Pharmaceutical aerosol composition and application thereof for treatment and prophylaxis of viral diseases. U.S. Pat. No. 5,723,439].

In 2010, a further method was proposed for an aerosol preparation for treating viral respiratory infections, in which the aerosol preparation comprises, as an active ingredient, APR in a quantity of from 23 to 30 mg per 100 ml of the preparation, 70-84% by volume of 1,1,1,2-tetrafluoroethane as a propellant, 8-15% by volume of ethanol, 5-10% by volume of glycerol and water as solvents, and a stabilizer. The aerosol preparation is adapted for use under pressure in an aerosol container with a metering value [Zhirnov, O. P.; Khanykov, A.V. Aprotinin-based for the aerosol preparation for the treatment of viral respiratory infection. International Patent Application WO 2012/008869, 2012. https://patentscope.wipo.int/search/en/detail.jsf?docId=WO2012008869. European Patent Application EP 2594283 B1].

An approximately 10-fold reduction in viral load was shown in patients treated with APR aerosol compared with patients treated with Ingavirin. The duration of clinical symptoms, such as rhinorrhea, weakness, headache, sore throat, cough, chest pain, and fever, was 1-2 days shorter in the APR group than in the Ingavirin group. Side effects and discomfort in patients of the APR group were not identified. [Zhirnov O. P. et al. Pathogenetic treatment of influenza patients with aerosolized form of aprotinin, a protease inhibitor. BIOpreparations. Prevention, Diagnosis, Treatment. 2015, (4), 59-64. https://www.biopreparations.ru/jour/article/view/35/36.].

Ectopic myocardial trypsin is known to be involved in acute and chronic myocardial inflammation by promoting infection with influenza A virus (IAV) and initiating the trypsin-MMP-9-cytokine cycle, as well as promoting progressive cardiac dilatation through collagen remodeling. Trypsin plays a vital role in the development of dilated cardiomyopathy (DCM) after IAV infection. Aprotinin has been shown to prevent the progression of myocarditis to DCM by inhibiting HAV infection, interrupting the trypsin-MMP-9-cytokine cycle, and restoring collagen metabolism. Thus, inhibition of trypsin activity is a promising therapeutic approach to prevent DCM following IAV infection [Pan, H. Y. et al. Up-regulation of ectopic trypsins in the myocardium by influenza A virus infection triggers acute myocarditis, Cardiovascular Research, 2011, 89, 595-603; doi: 10.1093/cvr/cvq358. Pan, H. Y. et al. Ectopic trypsin in the myocardium promotes dilated cardiomyopathy after influenza A virus infection. Am J Physiol Heart Circ Physiol, 2014, 307, H922-H932; doi: 10.1152/ajpheart.00076.2014. Shi, J. Y. et al. Expression of ectopic trypsin in atherosclerotic plaques and the effects of aprotinin on plaque stability. Arch Biochem Biophys. 2020, 690, 108460; doi: 10.1016/j.abb.2020.108460.].

Acute myocarditis is a well-known complication of influenza infection and a common prelude to inflammatory dilated cardiomyopathy (DCM) that can lead to chronic heart failure [Al-Amoodi, M. et al. Fulminant myocarditis due to H1N1 influenza. Circ Heart Fail. 2010, 3, e7-9; doi: 10.1161/CIRCHEARTFAILURE.110.938506. Ukimura, A. et al. Myocarditis Associated with Influenza A H1N1pdm2009. Influenza Res Treat. 2012, 2012, 351979. doi: 10.1155/2012/351979.].

The most well known complication of a SARS-CoV-2 infection is development of coronavirus disease (COVID-19). At least a third of people infected with SARS-CoV-2 do not have symptoms of COVID-19. [Oran, D. P.; Topol, E. J. The Proportion of SARS-CoV-2 Infections That Are Asymptomatic: A Systematic Review. Annals of Internal Medicine 2021, 174, 655-662. doi:10.7326/M20-6976.]. Of those who develop symptoms noticeable enough to be classified as patients, most (81%) develop mild to moderate symptoms (up to mild pneumonia), 14% develop severe symptoms (dyspnea, hypoxia, or more than 50% lung involvement, and 5% develop critical symptoms (respiratory failure, shock, or multiorgan dysfunction) [CDC. Interim Clinical Guidance for Management of Patients with Confirmed Coronavirus Disease (COVID-19). Jun. 30, 2020.].

APR is the most interesting drug candidate for preventing and treating SARS-CoV-2/COVID-19. It is not only an inhibitor of SARS-CoV-2 entry into the host cell [Bojkova, D. et al. Aprotinin Inhibits SARS-CoV-2 Replication. Cells 2020 9, 2377. doi:10.3390/cells9112377.], but also as an inhibitor of coagulation (clot formation) and an effective anti-inflammatory drug. [Didiasova, M. et al. Factor XII in coagulation, inflammation and beyond. Cell Signal. 2018, 51:257-265. doi:10.1016/j.cellsig.2018.08.006. Taylor K. M. Anti-inflammatory effects of aprotinin. Transfus. Altern. Transfus. Med., 2004, 6 (4), 39-46. doi: 10.1111/j.1778-428X.2004.tb00236.x]. Indeed, APR has been termed a “broad-spectrum antifibrinolysin” due to its anti-inflammatory and endotheliomodulatory effects [Peters D C, Noble S. Aprotinin: an update of its pharmacology and therapeutic use in open heart surgery and coronary artery bypass surgery. Drugs 57, 233-260 (1999). doi:10.2165/00003495-199957020-00015.].

APR has demonstrated efficacy in pre-and post-exposure prophylaxis of SARS-CoV-2. Efficacy as a prophylactic treatment of COVID-19 has been shown in a hamster model and verified in a hospital setting during the COVID-19 pandemic [Ivashchenko, A. A. et al. Aprotinin—a New Drug Candidate for The Prevention of SARS-CoV-2 (COVID-19). 2020. COVID19-preprints.microbe.ru; doi 10.21055/preprints-3111813; https://covid19-preprints.microbe.ru/files/140].

An open, non-comparative study showed efficacy and safety in treating patients hospitalized with COVID-19 with intravenous and inhaled APR [Ivashchenko, A. A. et al. Effect of Aprotinin and Avifavir® Combination Therapy for Moderate COVID-19 Patients. Viruses 2021, 13, 1253. https://doi.org/10.3390/v13071253].

The efficacy of APR has also been confirmed in a randomized phase III treatment of hospitalized patients with mild to moderate SARS-CoV-2 infection and moderate COVID-19 pneumonia [Redondo-Calvo, F. J. et al. Aprotinin treatment against SARS-CoV-2: A randomized phase III study to evaluate the safety and efficacy of a pan-protease inhibitor for moderate COVID-19. Eur J Clin Invest. 2022, 52, e13776; doi: 10.1111/eci.13776. Redondo-Calvo, F. J. et al. Inhaled aprotinin reduces viral load in mild-to-moderate in patients with SARS-CoV-2 infection. Eur J Clin Invest. 2022, 52, e13850; doi: 10.1111/eci.13850].

Liposomal formulations made their successful entry into the market in 1995. Liposomal delivery systems have been explored for diseases ranging from cancer treatment to pain management. Several liposomes have been successfully applied in the clinic, and other liposomal preparations are at different stages of clinical research [Bulbake, U. et al. Liposomal Formulations in Clinical Use: An Updated Review. Pharmaceutics 2017, 9, 12; https://doi.org/10.3390/pharmaceutics9020012.].

The present application relates in part to liposomal formulations of APR. Research on Lip-APR and similar liposomal formulations is limited to only a few studies. In one study, the primary inhibitor of pancreatic proteinases, BPTI, was formed in complexes with multilamellar vesicles (MLV) from six different preparations of soybean phospholipids with varying compositions. However, the antiproteinase activity of BPTI in these complexes was initially relatively low. This activity increased to 70% of its original level when adding sodium deoxycholate. [Tiourina, O. P. et al. Complexing of Basic Pancreatic Proteinase Inhibitor with Soybean Phospholipid Multilamellar Vesicles. Biochemistry (Moscow) 2001, 66, 340-344; https://doi.org/10.1023/A: 1010268317266.].

A study investigating the treatment of hypoxemia in rats with acute lung injury using Lip-APR (liposomes derived from lycetin and cholesterol-containing APR) was carried out under conditions of an experimental model of acute lung injury by intratracheal administration of acidin-pepsin to rats. This study showed that the lethality of treated experimental animals did not change compared to controls after treatment with Lip-APR [Kulikov, O A Evaluation of the effect of liposomal aprotinin on the degree of hypoxemia in the rats with acute lung injury. The Journal of scientific articles “Health and Education Millennium,” 2017, 19 (9), 155-157 (Russ).

https://cyberleninka.ru/article/n/otsenka-vliyaniya-liposomalnoy-formy-aprotinina-na-stepen-gipoksemii-u-krys-pri-ostrom-povrezhdenii-lyogkih/viewer.].

Despite the positive impacts of APR in the treatment of RNA virus infections, there remains a need for more effective therapeutics with low side effects. Thus, the inventors of the present application have developed new compositions, pharmaceuticals, formulations, and methods to treat and prevent RNA viral infections in mammals and diseases associated with such infections.

### BRIEF DESCRIPTION OF THE DRAWINGS

FIG. 1 depicts confluent hemorrhagic pneumonia in the lungs of hamsters with COVID-19 infection on day 6 after infection in control group #1 (A) and treatment group #2 (C) in Example 4.

FIG. 2 depicts survival (A) and change in body weight (B) of mice treated intraperitoneally with Lip-APR when infected with influenza A/California/04/2009 (H1N1) in Example 5.

### TERMS USED IN THE DESCRIPTION

In the context of this application, the term “liposome” in its singular and plural form refers to vesicles composed of a bilayer (uni-lamellar) and/or a concentric series of multiple bilayers (multilamellar) separated by aqueous compartments formed by amphipathic molecules such as phospholipids that enclose a central aqueous compartment.

The term “Liposomal aprotinin” (Lip-APR) refers to liposomes containing aprotinin.

The term “pharmaceutical composition” refers to a composition comprising at least one active ingredient, e.g., Lip-APR, in which at least one of the excipients is selected from the group consisting of pharmaceutically acceptable and pharmacologically compatible fillers, solvents, diluents, carriers, excipients, distributing, and receptive agents, delivery agents such as preservatives, stabilizers, fillers, disintegrators, moisteners, emulsifiers, suspending agents, thickeners, sweeteners, flavoring agents, aromatizing agents, antibacterial agents, fungicides, lubricants, and prolonged delivery controllers or liposomes.

The term “excipient,” as used herein, refers to a compound used to prepare a pharmaceutical composition that is generally safe, non-toxic, and neither biologically nor otherwise undesirable and includes excipients acceptable to humans and animals.

The term “therapy” or “medical treatment” is the attempted remediation of a health problem, usually following a medical diagnosis.

The term “combination therapy” is a therapy that uses more than one medication or modality. Typically, the term refers to using multiple therapies to treat a single disease, and often all the therapies are pharmaceutical. “Pharmaceutical” combination therapy may be achieved by prescribing/administering separate drugs or, where available, dosage forms that contain more than one active ingredient (such as fixed-dose combinations).

The term “pharmaceutical combination therapy” is a therapy that uses at least two drugs. “Pharmaceutical combination therapy” may be achieved by prescribing/administering separate drugs or dosage forms that contain at least two active ingredients (such as fixed-dose combinations).

The term “parenteral therapies” are therapies in which the administration of drugs is primarily via injection (intravenously, into the muscles, under the skin), inhalation, and nasally (spray, drops).

The term “therapeutically effective amount” or “dose,” as used herein, means the amount of medicine needed to reduce the symptoms of a disease in a patient. The dose of medicine will be tailored to the individual requirements in each case. This dose can vary widely depending on numerous factors, such as the severity of the patient's illness, the age and general health of the patient, other drugs with which the patient is being treated, the method and form of administration of medicine, and the experience of the attending physician. Typically, treatment is started with a large initial “loading dose” to rapidly reduce or eliminate the virus, followed by tapering the dose to a sufficient level to prevent an outbreak of infection.

The term “patient” means a mammal, including but not limited to humans, cattle, pigs, sheep, chickens, turkeys, buffaloes, llamas, ostriches, dogs, cats, hamsters, and mice; preferably, the patient is a human.

The term “drug” (also called medicine, medicament, pharmaceutical drug, or medicinal drug) refers to a drug used to diagnose, cure, treat, or prevent disease and means a substance (or a mixture of substances in the form of a pharmaceutical composition).

The term “oral drug” refers to solutions, powders, tablets, capsules, and pills taken by mouth and swallowed.

The term “parenteral drug” refers to drugs administered into the body, bypassing the gastrointestinal tract. Examples of parenteral drugs are solutions for injection, inhalation, and sprays, including for nasal or drip application, and other finished dosage forms, in this case, intended for treating and preventing viral infections and diseases caused by them.

### SUMMARY OF THE INVENTION

The first aspect of the present application is a method for treating and preventing an RNA viral infection and a disease associated with the infection in mammals, comprising: administering to a patient a pharmaceutical composition containing liposomal aprotinin (Lip-APR) in a therapeutically effective amount.

In accordance with the present application, the pharmaceutical composition includes Lip-APR and water for injection in a therapeutically effective amount.

In accordance with the present application, the pharmaceutical composition includes Lip-APR, APR, and water for injection in a therapeutically effective amount.

In accordance with the present application, in a preferred embodiment, the Lip-APR comprises at least APR. phosphatidylcholine (Lipoid S100), cholesterol, distearoylphosphatidylethanolamine-(polyethylene glycol) (DSPE-PEG-2000).

The subject of the present application is also a method for treating and preventing an RNA viral infection and a disease associated with this infection in mammals, comprising: administering a pharmaceutical composition containing a therapeutically effective amount of aprotinin (APR).

In accordance with the present application, exemplary (non-limiting) examples of an RNA viral infection include influenza and an influenza-related disease or a coronavirus and a coronavirus-related disease (for example, SARS-CoV-2 virus and COVID-19).

In accordance with the present application, in one embodiment, a pharmaceutical composition containing Lip-APR is administered parenterally to a mammal.

In accordance with the present application, in one embodiment, a pharmaceutical composition containing Lip-APR and APR is administered parenterally to a mammal.

In accordance with the present application, parenteral administration is intravenous, inhalation, or nasal administration.

Another aspect of the present application is an anti-RNA viral pharmaceutical composition in the form of an aqueous dispersion or emulsion containing Lip-APR.

In accordance with the present application, an anti-RNA viral pharmaceutical composition comprises APR.

In accordance with the present application, in a preferred embodiment, the pharmaceutical composition comprises at least APR, phosphatidylcholine, cholesterol, distearoylphosphatidylethanolamine-(polyethylene glycol), and water for injection, wherein the APR is included in a therapeutically effective amount.

Another aspect of the present application is Lip-APR, comprising at least APR, phosphatidylcholine, cholesterol, and distearoylphosphatidylethanolamine-(polyethylene glycol) in a therapeutically effective amount.

Another aspect of the present application is the use of Lip-APR in a therapeutically effective amount for the treatment and prevention of RNA viral infection and disease associated with this infection in mammals.

Another aspect of the present application is the use of Lip-APR in a therapeutically effective amount for the treatment and prevention of influenza and influenza-associated diseases in mammals.

Another aspect of the present application is the use of Lip-APR in a therapeutically effective amount for the treatment and prevention of coronavirus and diseases associated with coronavirus infection in mammals.

Another aspect of the present application is the use of Lip-APR in a therapeutically effective amount for the treatment and prevention of SARS-CoV-2 and disease associated with SARS-CoV-2 in mammals.

Another aspect of the present application is the use of Lip-APR in a therapeutically effective amount for the treatment and prevention of COVID-19 in mammals.

Non-limiting exemplary embodiments of the present application and the efficacy of Lip-APR in treating an RNA viral infection and a disease associated with such infection in mammals are presented in Examples 1 to 5.

### EXAMPLE 1. PREPARATION OF A LIPOSOMAL PHARMACEUTICAL COMPOSITION COMPRISING LIP-APR

Stage 1. Obtaining a lipid film. We dissolved 99.0 g of phosphatidylcholine (Lipoid S100, Lipoid GmbH, Germany), 9.0 g of cholesterol ≥99% (Sigma-Aldrich, USA), and 2.1 g of distearoylphosphatidylethanolamine-(polyethylene glycol)-2000 (DSPE-PEG-2000, Lipoid GmbH, Germany) in 500 ml of chloroform for 20 minutes. The resulting solution was filtered using Chemical Duty Vacuum Pressure Pump supports (Merck Millipore WP6122050) through a nylon filter with a diameter of 47 mm and a pore size of 0.22 μm and transferred to a flask with a capacity of 20 liters. The organic solvent was distilled off under reduced pressure (250-300 mbar) until a translucent lipid film formed on the walls of the flask, which was finally dried at a pressure of 100-150 mbar for 2.5 hours.

Stage 2. Obtaining a sterile solution of aprotinin. 1.836 g of APR (powder) from Jiuquan Dadeli Pharm Co., Ltd., China, with an activity of 6210 KIU/mg (total 11,401,560. KIU of APR) was dissolved in 1080 ml of water for injection (saline) in a 2000 ml beaker. The resulting aqueous solution of APR was filtered through a Stericap filtration system with a pore size of 0.22 μm.

Stage 3. Hydration of the lipid film. The film was hydrated with a sterile saline solution of APR to obtain a dispersion of multilayer APR liposomes. The lipid film was washed off at a rotor speed of 20-30 rpm. The pressure in the system was reduced to 100 mbar 10 min before the end of the process to remove oxygen and other gases formed during hydration from the liposomal dispersion. The total hydration time was three hours.

Step 4: Obtaining an emulsion pharmaceutical composition comprising Lip-APR. The dispersion of multilayer liposomes was sequentially passed on an extruder through nylon filters with different pore diameters, from 1.2 to 0.22 μm (after 1.2 and 0.45, extrusion was performed one time, after 0.22-three times). Emulsion pharmaceutical compositions comprising Lip-APR were obtained, the compositions of which are presented in Table 1.

TABLE 1
Emulsion pharmaceutical compositions containing Lip-APR.

| ^{a}The content of components in | [symbol] Lip-APR composition | | | | |
|---|---|---|---|---|---|
| 1 ml of liposoial emulsion | 1 | 2 | 3 | 4 | 5 |
| Aprotinin, mg | 1.6 | 1.6 | 1.69 | 1.836 | 1.55 |
| Lipoid S100, mg | 91.0 | 91.0 | 91.7 | 91.7 | 91.7 |
| Cholesterol, mg | 10 | 10 | 8.3 | 8.3 | 8.3 |
| DSPE-PEG-2000, mg | 2 | 2 | 1.9 | 1.9 | 1.9 |
| Water for injection up to 1 mL, mg | 895.4 | 895.4 | 895.4 | 895.4 | 370.0 |
| Liposome diameter, nm | 220 ± 8 | 243 ± 10 | 205 ± 10 | 191 ± 24 | 217 ± 15 |
| ^{b}Genera contents of APR, KIU/mL | 9,378 | 9,936 | 10,495 | 11,402 | 9,180 |
| ^{b}APR incorporation into liposomes, | 8,505 (90.7) | 9,012 (90.7) | 7,431 (70.8) | 8,073 (70.8) | 6,354 (69.2) |
| KIU/mL (%) | | | | | |
| ^{b}Free APR, KIU/ml (%) | 873 (9.3) | 924 (9.3) | 3,064 (29.2) | 3,329 (29.2) | 2826 (30.8) |
| ^{b}Switching efficiency APR, % | 90.7 | 90.7 | 70.8 | 70.8 | 69.2 |

^{a}Received emulsion liposomal pharmaceutical compositions (250 ml-370 ml in 500 ml bottles).
^{b}Total APR content and free APR content in an emulsion pharmaceutical composition containing Lip-APR and APR was determined by titrimetry [European Pharmacopoeia Supplement 10.8. April 2021: 0579 Aprotinin concentrated solution, https://www.drugfuture.com/Pharmacopoeia/EP6/data/aprotinin%20concentrated%20solution.pdf.].

### EXAMPLE 2. PREPARATION OF LYOPHILIZED PHARMACEUTICAL COMPOSITIONS COMPRISING LIP-APR

Stage 1. Obtaining a lipid film. Dissolved 102.7 g of Lipoid S100 (Lipoid GmbH, Germany) or egg yolk phosphatidylcholine (Sigma-Aldrich, USA), 9.3 g of cholesterol ≥99% (Sigma-Aldrich, USA) and 2.1 g of DSPE-PEG-2000 (Lipoid GmbH, Germany) in 500 ml of chloroform for 20 minutes. The resulting solution was filtered using Chemical Duty Vacuum Pressure Pump supports (Merck Millipore WP6122050) through a nylon filter with a diameter of 47 mm and a pore size of 0.22 μm and transferred to a flask with a capacity of 20 liters. The organic solvent was distilled off under reduced pressure (250-300 mbar) until a translucent lipid film formed on the walls of the flask, which was finally dried at a pressure of 100-150 mbar for 2.5 h.

Stage 2. Obtaining a sterile solution of April 1.9 g of APR (powder) from Jiuquan Dadeli Pharm Co., Ltd., China, with an activity of 6210 KIU/mg (total 11,799,000. KIU of APR) was dissolved in 1120 ml of saline for injection in a 2000 ml beaker. The resulting aqueous solution of APR was filtered through a Stericap filtration system with a pore size of 0.22 μm.

Stage 3. Hydration of the lipid film. The film was hydrated with a sterile solution of APR to obtain a dispersion of multilayer APR liposomes. The lipid film was washed off at a rotor speed of 20-30 rpm. The pressure in the system was reduced to 100 mbar 10 min before the end of the process to remove oxygen and other gases formed during hydration from the liposomal dispersion. The total hydration time was three hours.

Step 4: Obtaining lyophilizate of Lip-APR. The dispersion of multilayer liposomes was sequentially passed on an extruder through nylon filters with different pore diameters, from 1.2 to 0.22 μm (after 1.2 and 0.45, extrusion was performed one time, after 0.22-3 times). The volume of the liposomal dispersion was determined, and the required amount of sucrose was added to obtain a 10% sucrose dispersion of liposomes. The dissolution was carried out with uniform stirring on a shaker for one hour. Another extrusion cycle was carried out through filters with a pore size of 0.22 μm to sterilize the finished dispersion.

The resulting sterile liposomal emulsion was filled in 5 ml aliquots into 20 ml colorless neutral glass vials of hydrolytic class I using a dispenser, and the vials were placed in an Edwards Minifast DO.2 freeze dryer for lyophilization. The vials were cooled to −45° C. for 3 hours, then kept at −45° C. for 4.0-4.5 hours, and then the vacuum pump was turned on and kept at reduced pressure and a temperature of −45° C. for another 3 hours. Then the temperature in the freeze dryer was raised: to −20° C. at a rate of +5° C./h and kept at this temperature for 2 hours; then to −4° C. at a rate of +2° C./h. After overcoming the eutectic point (−6 to −9° C.), the heating rate was increased to +5° C./h. Residual moisture was removed by final drying of the preparation at a temperature above 0° C. To do this, the temperature in the freeze dryer was raised to +22° C. at and kept until room temperature was reached. The endpoint for drying the lyophilizate of Lip-APR was determined by achieving a constant value of the residual steam pressure in the freeze dryer. At the end of drying, the vacuum in the freeze dryer was quenched with clean, filtered air, which entered the chamber through a filter system. The total duration of lyophilization of liposomal aprotinin averaged 50 hours. The vials with Lip-APR were sealed with rubber stoppers and crimped with aluminum caps (Table 2).

TABLE 2
Lyophilized pharmaceutical compositions comprising Lip-APR.
^{a}Composition per 1 bottle

| of lyophilized liposomal | [symbol] Lip-APR composition | |
|---|---|---|
| pharmaceutical compositions | 6 | 7 |
| Aprotinin, mg | 8.5 | 8.5 |
| Lipoid S100, mg | 0 | 459 |
| Egg yolk phosphatidylcholine, mg | 500 | 0 |
| Cholesterol, mg | 33.5 | 42 |
| DSPE-PEG-2000, mg | 11 | 10 |
| Sucrose, mg | 500 | 500 |
| Liposome diameter, nm | 203 ± 11 | 212 ± 10 |
| ^{b}General contents of APR, KIU/bottle | 122,188 | 72,000 |
| ^{b}APR incorporation into liposomes, | 52,785 (43.2) | 49,680 (69.0) |
| KIU/bottle (%) | | |
| ^{b}Free APR, KIU/bottle (%) | 69.40 (56.8) | 22.32 (31.0) |

^{a}Received a liophilisate of liposomal pharmaceutical compositions in 20 ml bottles.
^{b}Total APR content and free APR content in an emulsion pharmaceutical composition containing Lip-APR and APR was determined by titrimetry [European Pharmacopoeia Supplement 10.8. April 2021: 0579 Aprotinin concentrated solution, https://www.drugfuture.com/Pharmacopoeia/EP6/data/aprotinin%20concentrated%20solution.pdf.]

### EXAMPLE 3. INTRAVENOUSLY TREATMENT OF SYRIAN HAMSTERS INFECTED WITH THE SARS-COV-2 WITH LIP-APR

The efficacy of an embodiment of the Lip-APR drug of the present application was evaluated using a model of SARS-CoV-2 infection in Syrian hamsters [R. Boudewijns et al. STAT2 signaling as double-edged sword restricting viral dissemination but driving severe pneumonia in SARS-CoV-2 infected hamsters. BioRxiv preprint. doi: https://doi.org/10.1101/2020.04.23.056838; this version posted Apr. 24, 2020. https://www.biorxiv.org/content/10.1101/2020.04.23.056838v1].

The study used severe acute respiratory syndrome coronavirus 2 isolate SARS-CoV-2/human/AUS/VIC01/2020 (GenBank: MT007544.1)[https://www.ncbi.nlm.nih.gov/nuccore/MT007544].

Wild-type Syrian hamsters at the age of 6-10 months weighing 100-120 g were kept with unlimited access to food and water. Hamsters were randomized into 7 groups:
Group 1—control group, untreated hamsters (n=8). Dose: 5 ml/kg saline.
Group 2—treatment male Syrian hamsters (n=4) with APR-Dose: 50,000 KIU/kg APR.
Group 3—treatment male Syrian hamsters (n=4) with Lip-APR-Dose: 50,000 KIU/kg APR (composition [symbol] 2, Example 1, Table 1).
Group 4—treatment female Syrian hamsters (n=4) with APR-Dose: 50,000 KIU/kg APR.
Group 5—treatment female Syrian hamsters (n=4) with Lip-APR-Dose: 50,000 KIU/kg APR (composition [symbol] 2, Example 1, Table 1).

Hamsters were anesthetized with zoletil-xyla and inoculated into each nostril with 50 μl anesthetic combination containing 10³TCID₅₀.

The drugs were injected under light isoflurane anesthesia intravenously, 2 times a day for 4 days, starting the first injection one hour before infection, 6 hours after infection, then for 3 days after 12 hours.

Hamsters were checked daily for appearance, behavior, and weight. 96 hours after infection, the hamsters were euthanized by intravenous injection of 500 μl doletal (200 mg/ml sodium pentobarbital, Vétoquinol SA). Hamster lung tissues were harvested after sacrifice and homogenized using a Precellys homogenizer in a 350 μl RNeasy lysis buffer (RNeasy Mini kit, Qiagen) and centrifuged (10,000 rpm, 5 min) to remove cell debris. RNA was extracted according to the manufacturer's instructions. Real-time PCR was performed on the LightCycler96 platform (Roche) using the iTaq Universal Probes One-Step RT-qPCR kit (BioRad) [R. Boudewijns et al. STAT2 signaling as double-edged sword restricting viral dissemination but driving severe pneumonia in SARS-CoV-2 infected hamsters. BioRxiv preprint. doi: https://doi.org/10.1101/2020.04.23.056838; this version posted Apr. 24, 2020. https://www.biorxiv.org/content/10.1101/2020.04.23.056838v1].

For histological analysis, lung tissue was fixed in 4% formaldehyde, embedded in paraffin, and stained with hematoxylin-cosin. Damage was assessed on a scale from 1 to 3: stagnation, intra-alveolar bleeding, apoptotic bodies in the bronchial epithelium, necrotic bronchiolitis, perivascular edema, bronchopneumonia, perivascular inflammation, peribronchial inflammation, and vascular inflammation.

Statistical analysis was performed using the GraphPed Prism software from GraphPed Software, Inc. Statistical significance was determined using the Mann-Whitney nonparametric U-test. The values of P≤0.05 were considered significant.

An analysis of the results showed that the Lip-APR group demonstrated a high anti-SARS-CoV-2/COVID-19 efficacy (p<0.05), while under comparable conditions, the control group of untreated hamsters and the group treated with APR showed no antiviral activity (Table 3, Table 4 and Table 5).

In the treatment of SARS-CoV-2 infected animals, the infectious titer (lgTCID50/ml) in the lungs of infected animals in the control groups was 4.60±0.42 (male), 3.90±0.82 (female), 4.25±0.72 (male+female) and the APR groups was 3.88±0.48 (male), 4.38±0.85 (female), 4.13±0.69 (male+female). However, the p-value (p>0.05) for the APR groups indicated the absence of the antiviral effect of APR under the studied conditions. The infectious titer of Lip-APR groups was 3.38±0.48 (male), 3.50±0.50 (female), and 3.43±0.45 (male+female). At the same time, in all Lip-APR groups, there was a significant decrease in the infectious titer (p<0.05), which significantly exceeded the reduction of infectious titer in the control group [symbol] 1.

In the treatment of SARS-CoV-2 infected animals, the viral load (Ct) in the lungs of infected animals in the control groups was 16.34±0.86 (male), 19.36±3.78 (female), 17.85±3.03 (male+female) and the APR groups was 17.51±1.28 (male), 17.51±1.04 (female), 17.72±1.10 (male+female). However, the p-value (p>0.05) for the APR groups indicated the absence of the antiviral effect of APR under the studied conditions. The infectious titer of Lip-APR groups was 20.61±1.72 (male), 21.86±2.62 (female), and 21.14±2.05 (male+female). At the same time, in all Lip-APR groups, there was a significant increase in the viral load (p<0.05), which significantly exceeded the increase of the viral load in the control group [symbol] 1.

TABLE 3
Viral load (Ct) and infectious titer values (TCID₅₀/mL) of lung
homogenates from male Syrian hamsters treated with Lip-APR.

| | | | | | Infectious titer | |
|---|---|---|---|---|---|---|
| | | Dose, | Viral load | | lg (TCID₅₀/ | |
| Group | Drug | KIU/kg | Ct ± SD | p** | mL) ± SD | p* |
| 1 | Saline | 0 | 16.34 ± 0.86 | | 4.60 ± 0.42 | |
| 2 | APR | 50,000 | 17.51 ± 1.28 | 0.191 | 3.88 ± 0.48 | 0.079 |
| 3 | Lip-APR* | 50,000 | 20.61 ± 1.72 | 0.016 | 3.38 ± 0.48 | 0.024 |

*Composition #2, Example 1, Table 1.
**p ≤ 0.05—having an antiviral effect, p ≥ 0.05—no antiviral effect.

TABLE 4
Viral load (Ct) and infectious titer values (TCID₅₀/mL) of lung
homogenates from female Syrian hamsters treated with Lip-APR.

| | | Dose, | Viral load | | Infectious titer | |
|---|---|---|---|---|---|---|
| Group | Drug | KIU/kg | (Ct ± SD) | p** | lg (TCID₅₀/mL) ± SD | p* |
| 1 | Saline | 0 | 19.36 ± 3.78 | | 3.90 ± 0.82 | |
| 4 | APR | 50,000 | 17.51 ± 1.04 | 0.730 | 4.38 ± 0.85 | 0.635 |
| 5 | Lip-APR* | 50,000 | 21.86 ± 2.62 | 0.036 | 3.50 ± 0.50 | 0.679 |

*Composition #2, Example 1, Table 1.
**p ≤ 0.05 - having an antiviral effect, p ≥ 0.05 - no antiviral effect.

TABLE 5
Viral load (Ct) and infectious titer values (TCID₅₀/mL) of lung homogenates
from male and female Syrian hamsters treated with Lip-APR.

| | | Conc.*, | Dose, | Viral load | | Infectious titer | |
|---|---|---|---|---|---|---|---|
| Group | Drug | KIU/ml | KIU/kg | Ct ± SD | p** | lg (TCID₅₀/mL) ± SD | p* |
| 1 | Saline | 0 | 0 | 17.85 ± 3.03 | | 4.25 ± 0.72 | |
| 6 (2 + 4) | APR | 10,000 | 50,000 | 17.72 ± 1.10 | 0.360 | 4.13 ± 0.69 | 0.483 |
| 7 (3 + 5) | Lip-APR | 10,000 | 50,000 | 21.14 ± 2.05 | 0.033 | 3.43 ± 0.45 | 0.017 |

*Drug concentration.
**p ≤ 0.05—having an antiviral effect, p ≥ 0.05—no antiviral effect.

The above results of intravenous treatment of SARS-CoV-2 infected Syrian hamsters with Lip-APR (Example 3) confirm the high efficacy of Lip-APR compared to control and APR. Treatment with Lip-APR effectively prevented the clinical signs of the disease, suppressed the reproduction of the virus and the development of pneumonia in the lungs.

### EXAMPLE 4. INHALATION TREATMENT WITH LIP-APR IN SYRIAN HAMSTERS INFECTED WITH THE SARS-COV-2 VIRUS

The study used the SARS-CoV-2 coronavirus strain (GenBank ID: MW161041.1) [https://www.ncbi.nlm.nih.gov/nuccore/MW161041.1].

All animals in the experiments were infected with the SARS-CoV-2 virus intranasally under light ether anesthesia in a volume of 100 μl for both nostrils. In both experiments, an infection dose of 10^{3.7} TCID₅₀ was used. The animals were monitored daily throughout the duration of the experiment.

In the experiment, saline and Lip-APR (Composition [symbol] 5 from example 1, 9180 KIU/ml APR) were used as drugs.

The experiment involved 2 groups of 5 female Syrian hamsters, from one batch, weighing 50-60 g each.
Group 1 (control)—inhalation administration of saline.
Group 2 (treatment group)—inhalation administration of Lip-April 3 times per day.

In total, the Syrian hamsters received 3 inhalations (morning, afternoon and evening) of saline or Lip-APR:
on the 1st day, the animals received the drug 3 hours after infection (12 pm) with the SARS-CoV-2 virus, then the animals received another dose in the evening (9 pm);
over the next 5 days (total 6 days of treatment), the animals received the drug 3 times a day at 9 am, 3 pm and 9 pm.

The study drug was administered by inhalation using a nebulizer. For inhalation, a group of 5 animals was placed in a chamber and administered 3 ml (9180 KIU/ml, total 27540 KIU, 5508 KIU/animal, 16520 KIU/animal/day) of the study drug for 10 minutes. Animals from the control group were administered saline via inhalation according to the same scheme and in the same volume.

Body weight was assessed daily, and temperature was measured. The virus titer in the lungs was determined in 5 hamsters in each group on the 6th day.

On day 6 after infection with the virus, hamsters in each group were sacrificed, and the lungs were removed under sterile conditions. A visual pathomorphological study of the lungs was conducted. After that, the lungs were homogenized and resuspended in 1 ml of cold sterile PBS solution. The suspension was cleared of cell debris by centrifugation at 2000 g for 10 min, and the supernatant was used to determine the infectious titer of the virus in the cell culture.

Euthanasia (painless killing of the animal) was carried out by a responsible person in accordance with the requirements adopted at the institute by dislocation of the cervical vertebrae with preliminary anesthesia with ether. Euthanasia was carried out without causing suffering.

By day 6, animals in virus control group #1 began to show clinical symptoms of a respiratory infection: severe recurrent sneezing, short-term coughing, nasal secretion, mild hyperemia of the mucous membranes, and a slight decrease in physiological activity.

In the Lip-APR inhalation therapy group #2, the animals did not show clinical symptoms of respiratory infection, such as sneezing, coughing, and decreased physiological activity throughout the observation period.

Evaluation of clinical signs of infection in animals in group No. 1 (virus control) and group No. 2 (treatment group) was carried out on a scoring system, as presented in Table 6. The results in the Table show that group No. 1 (virus control) exhibited a total severity of clinical signs of the disease at an estimated 12 points, whereas group No. 2 (treatment group) exhibited a total severity of 1 point (Table 6).

TABLE 6
Assessment of clinical signs of infection during inhalation
drug treatment in hamsters with COVID-19 infection.

| | Virus | Treatment |
|---|---|---|
| | control | with Lip-APR |
| | group [symbol] 1 | group [symbol] 2 |
| Sternutation (act of sneezing)^{a} | 4 | 0 |
| Cough act^{b} | 2 | 0 |
| The secretion of the nasal glands^{c} | 3 | 0 |
| Hyperemia of the mucous membranes^{d} | 2 | 1 |
| General animal activity^{e} | 1 | 0 |
| Sum: | 12 | 1 |

^{a}Sternutation (act of sneezing): absent - 0, single, repeated less than 1 time in 10 minutes - 1, single, repeated 1 or more times in 10 minutes - 2, multiple attacks following each other, repeated ~1 time in 10 minutes - 3, multiple attacks following one after another, repeated more often 2 times in 10 minutes - 4;
^{b}Cough act: absent - 0, single, repeated less than 1 time in 10 minutes - 1, single, repeated 1 and more often than once every 10 minutes - 2, multiple attacks following each other, repeated ~1 time in 10 minutes - 3, multiple attacks following each other, repeated more often 2 times in 10 minutes - 4;
^{c}Secretion of the nasal glands: secretion of the glands is normal (the mucous membrane is semi-moist, without a drop of exudate, the natural openings are dry) - 0, a slight increase in the secretion of the glands (the mucous membrane is moist with small droplets of exudate, rare small drops of exudate around the natural openings) - 1, moderate increase in gland secretion (the mucous membrane is moist, with large drops of exudate, a small amount of transparent exudate on the wool around the natural openings) - 2, hypersecretion of the glands (the mucous membrane is wet, swollen, around the natural openings there is a transparent exudate on the wool, from which it is wet, stuck together) - 3;
^{d}Hyperemia of the mucous membranes of the nasal and oral cavities: normal (pale pink color) - 0, slight hyperemia of the mucous membranes (pinkish color) -1, moderate hyperemia of the mucous membranes (bright red color) - 2, increased hyperemia of the mucous membranes (dark cherry color) - 3;
^{e}Physiological activity: normal (animals show a pronounced interest in water, food, irritants, easily adapt to changing environments) - 0, weakened (animals show moderate interest in water, food, respond slowly to stimuli, within a few seconds, adapt quickly to a change of scenery) - 1, moderately slow (animals show moderate interest in water, food, respond to stimuli reluctantly, the first seconds can be lost when the environment changes) - 2, inhibited (animals are little interested in food and water, respond poorly to stimuli, do not show interest in the environment when the environment changes) - 3.

Daily weighing of the animals showed no significant weight loss or gain in either the treated or virus control groups. An analysis of the body weight of animals using the Kruskal-Wallis method on days 4 and 5 of infection, when the animals showed clinical signs of the disease, showed no statistically significant difference between the groups of treated animals compared with the virus control group.

Throughout the experiment, the body temperature of hamsters, both in the viral control group and in the group of treated animals, was within the physiological norm (37.5-38.5). The animals' temperature was measured by the rectal method, in the morning, before the procedures to exclude falsely elevated body temperatures associated with the stress factor. Due to inflammatory processes associated with coronavirus infection in the body of hamsters, thermometry showed a slight increase in temperature. However, this increase was not manifested clinically (fever, tremor, redness of the mucous membranes, and skin epithelium) and was within the physiological norm in all experimental animals.

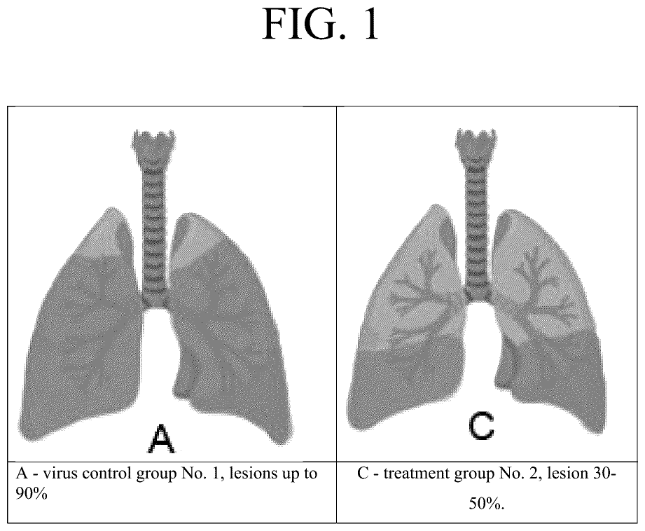

Visual pathoanatomical examination of the lungs extracted from animals on the 6th day after infection revealed serous-hemorrhagic edema of all lobes of the lungs and pronounced alterative-inflammatory changes in the virus control group. Additionally, extensive diffuse and punctate hemorrhagic confluent pneumonia was noted in this group, occupying up to 90% of the total lung volume, common both in the central regions of the lungs and in the marginal zones. Furthermore, partially emphysematous areas with increased pneumatization were found, and emphysema reached sizes from small areas with a modified structure to large pneumatized areas, characterized by the destruction of the walls of the alveoli and their fusion. Visual examination data were confirmed by virological studies, which showed that the virus titer in this group was high, averaging 5.6 lg TCID50/0.1 ml (FIG. 1, Table 6, 7).

With respect to group [symbol] 2, a visual pathoanatomical examination of the lungs revealed punctate and diffuse hemorrhagic pneumonia, occupying 30-50% of the total lung volume, equally distributed over the entire area of the lobes. No areas with emphysematous lesions were identified by visual assessment. Virological examination of the virus titer showed that the titer was lower than in the viral control group (4.3 lgTCID₅₀/0.1 ml vs 5.6 lgTCID₅₀/0.1 ml) (Table 6, 7).

TABLE 7
Virus titer in the lungs of hamsters with SARS-CoV-2 infection on
the 6th day after infection during inhalation drug treatment Group.

| Group | [symbol] [symbol] (lg [symbol] 50/0, 1M [symbol] ) |
|---|---|
| Virus control group [symbol] 1 | 5.5; 4.83; 4.5; 7.5; 5.83 (5.6) |
| (Saline) | |
| Treatment group #2 (Lip-APR) | 4.5; 4.17; 4.5; 4.5; 4.17 (4.3) |

The above results demonstrate inhaled treatment with Lip-APR in SARS-CoV-2-infected Syrian hamsters (Example 3) was effective. This treatment prevented clinical signs of the disease and suppressed the reproduction of the virus and the development of pneumonia in the lungs.

### EXAMPLE 5. INTRAPERITONEAL TREATMENTS OF MICE INFECTED WITH INFLUENZA A/CALIFORNIA/2009 (H1N1) PDM09 VIRUS WITH A PHARMACEUTICAL COMPOSITION INCLUDING LIP-APR IN AN INFLUENZA PNEUMONIA MODEL

In the experiment, 2 groups of BALB/c female mice weighing 12-14 g were formed: 1 group for intraperitoneal administration of Lip-APR and 1 control saline group, with 10 animals per group, of which 7 mice were tested for survival and 3 mice were tested for the titer of the virus in the lungs.

Intraperitoneal administration—Group 1 (Control)—Untreated Mice: On the morning of day 1, mice immediately after infection with influenza A/California/2009(H1N1) pdm09 were intraperitoneally injected with saline; mice then received a second dose of saline 8-12 hours later; then, day 2-5, mice received intraperitoneally 2 times a day.

Intraperitoneal administration—Group 2 (treated group)—treatment of mice with Lip-APR: on the morning of the 1st day immediately after infection with influenza A/California/2009(H1N1) pdm09. Lip-APR was administered intraperitoneally; then, day 2-5, mice were intraperitoneally injected 2 times a day of Lip-APR.

Mice randomized into groups were infected under light anesthesia at a dose of 5 MLD50/ml (25 μl per nostril, 104.5 TCID50/0.1 ml) intranasally adapted to mice with influenza A/California/2009 (H1N1) pdm09 virus obtained from WHO.

As indicated herein, saline was used in the control group, and a solution of Lip-APR in saline (9180 KIU/ml, Composition [symbol] 5 from example 1) was used a treatment group.

Saline and Lip-APR in saline was administered by intraperitoneal by 0.2 ml (1826 KIU) 2 twice a day (3652 KIU/day).

The treated and control animals were monitored daily for 16 full days (from the moment the animals were infected with the influenza virus). Mortality was recorded daily in both groups.

Euthanasia (painless killing of the animal) was carried out by the responsible person in accordance with the existing ethical requirements by dislocation of the cervical vertebrae with preliminary anesthesia with ether. Euthanasia was performed promptly after the end of the experiments.

The activity of the compounds in the mouse model of influenza pneumonia was assessed according to the following criteria: animal survival, average life expectancy, dynamics of weight loss, and lung virus titers after day 4 post-infection.

Mortality was defined as the ratio of dead to infected in a group of animals.

The average life span of animals was calculated from the total number of observation days (after infection) according to the formula: MSD=Σf*(d−1)/n, where f is the number of mice that died on day d; surviving mice, for which day d is the last day of observation, are also taken into account; and n is the number of mice in the group.

A statistically significant increase in the survival rate of animals (p<0.05), an increase in their lifespan, and a statistically significant decrease in the viral titer in the lungs of infected animals after the introduction of drugs was observed as compared to the control group of infected but untreated animals.

The digital data obtained was statistically processed using the Statistica 8.0 software. Comparison of survival in the groups of mice was performed by means of one-way analysis of variance (ANOVA) using the Statistica 8.0 software.

Evaluation of the weight of animals. Mice were weighed before the introduction of the test substances every other day. The decrease or increase in weight was calculated separately for each mouse and expressed as a percentage. In this case, the weight of the animal before infection was taken as 100%. For all mice of one group, the average value of the percentage of weight loss or weight gain was determined.

Obtaining mouse lung samples for study and determination of viral titer. On the 4th day after infection with the influenza virus, 3 mice in each group were euthanized, and their lungs were removed under sterile conditions, homogenized, and resuspended in 1 ml of cold sterile 0.01M PBS. The suspension was cleared of cell debris by centrifugation at 2000 g for 10 min. 0.1 ml of the supernatant was used to determine the infectious titer of the virus. To determine the infectious titer of the virus, MDCK cells were seeded in 96-well plates from Costar with an average density of 30000-35000 cells per well and grown in Eagle's minimum medium (MEM) in the presence of 5% fetal calf serum, 10 mM glutamine and antibiotics (penicillin 100 IU/ml and streptomycin 100 μg/ml) until complete monolayer. Before infection with the virus, the cells were washed twice with MEM without serum. 10-fold dilutions of each virus sample from the lungs (whole to 10−8) were prepared on a medium supplemented with TRNC-trypsin (2 μg/ml). The obtained dilutions infected the monolayer of 4 wells of a 96-well plate. After incubation at 37° C. in an atmosphere of 5% CO₂ for 72 hours, the cytopathic effect of the virus (CPE) was assessed (cytopathic infectious dose 50). Then the average value of the titer for 3 identical samples was calculated.

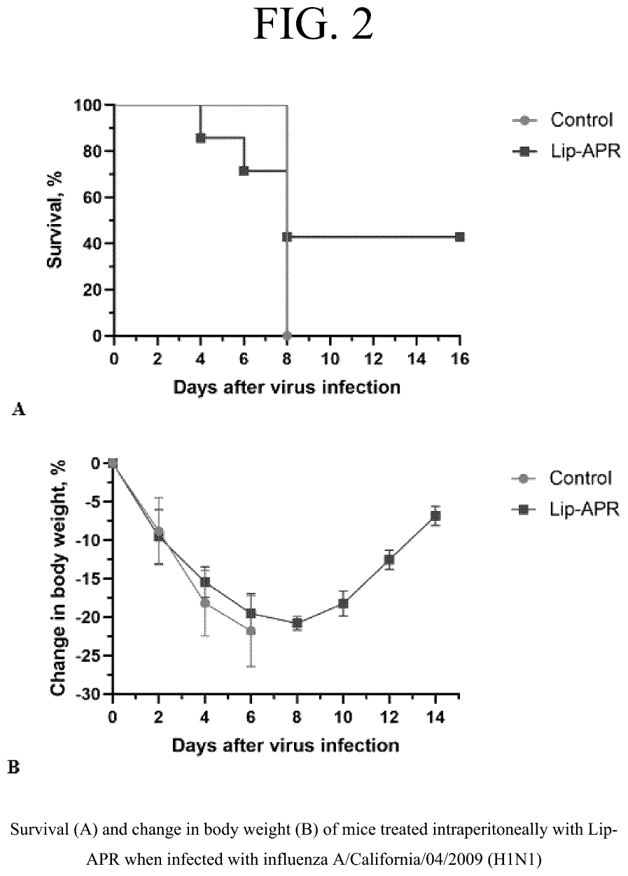

The results of intraperitoneal treatment with Lip-APR of infected mice of group No. 2 and control group No. 1 are presented in table 8 and FIG. 2.

TABLE 8
Results of intraperitoneal treatment with Lip-APR in mice infected
with mouse-adapted influenza A/California/04/2009 (H1N1) virus.

| | | | Average life | Virus titer, 4 days |
|---|---|---|---|---|
| | | | expectancy | post-infection |
| Groups | A drug | Survival, % | (days) | lgTCID₅₀/0.1 ml |
| 1 | Saline (virus | 100 | 7.0 | 5.25 ± 0.25 |
| | control) | | | (5.25; 5.5; 5.0) |
| 2 | Lip-APR | 58 | 9.5 | 5.25 ± 0.25 |
| | | (p = 0.05) | | (5.0; 5.50; 5.25) |

In the virus control group #3 (Table 9), all mice died on day 8 post-infection, and the mean lifespan in the virus control group was 7.0 days. Virus challenge also caused significant weight loss in mice (about 25% on study day 7) (FIG. 2, Table 8). A visual examination of the lungs of animals taken on the 4th day after infection to determine the virus titer in them revealed diffuse extensive hemorrhagic pneumonia, occupying up to 100% of the total lung volume, which was equally distributed in the central parts of the lungs and in the marginal zones. The pleura was tense, the volume of the lungs increased by an average of 1.5 times in contrast to the norm. Virological studies have shown that the titer of the virus in the lungs on day 4 after infection with the influenza A/California/04/09 (H1N1) virus was 5.25 lgTCID50/0.1 ml.

Intraperitoneal administration of Lip-APR protected 42% of animals from death, increased life expectancy by 1.4 times, and reduced weight loss (11% vs 25% in the viral control group). Visual examination of the lungs on the 4th day after infection revealed diffuse extensive hemorrhagic pneumonia, up to 80% of the total lung volume. One animal had an area of the lung with increased airiness-emphysema. Determination of the lung virus titer of animals in this group showed that it did not differ from that in the virus control group (5.25 lgTCID50/0.1 ml) (Table 8, FIG. 2).

As can be seen from Table 8 and FIG. 2, Lip-APR effectively protected mice from death and increased their life expectancy compared to the control group.

The preceding description of preferred embodiments has been presented for illustration and description only. It is not intended to be exhaustive or to limit the application to the precise form disclosed, and modifications and variations are possible and/or would be apparent in light of the above teachings or may be acquired from the practice of the application. The embodiments were chosen and described to explain the application's principles and its practical application to enable one skilled in the art to utilize the application in various embodiments and with various modifications suited to the particular use contemplated. It is intended that the scope of the application be defined by the claims appended hereto and that the claims encompass all embodiments of the application, including the disclosed embodiments and their equivalents.

We claim:

1. A method for treating and preventing an RNA viral infection and a disease associated with this infection in mammals, comprising:
administering to a patient in need thereof a pharmaceutical composition containing liposomal aprotinin (Lip-APR) in a therapeutically effective amount.

2. The method according to claim 1,
wherein the pharmaceutical composition comprises Lip-APR and water for injection in a therapeutically effective amount.

3. The method according to claim 1,
wherein the pharmaceutical composition comprises Lip-APR, APR outside of the liposomes, and water for injection in a therapeutically effective amount.

4. The method according to claim 1,
wherein the liposomes comprising the Lip-APR are composed of phosphatidylcholine (Lipoid S100), cholesterol, distearoylphosphatidylethanolamine-(polyethylene glycol) (DSPE-PEG-2000) in a therapeutically effective amount.

5. The method according to claim 1,
wherein the pharmaceutical composition further comprises a therapeutically effective amount of aprotinin (APR).

6. The method according to claim 1,
wherein the RNA viral infection is influenza and the disease associated with this infection is an influenza-related disease.

7. The method according to claim 1,
wherein the RNA viral infection is a coronavirus and the disease associated with this infection is a coronavirus-related disease.

8. The method according to claim 7,
wherein the coronavirus is SARS-CoV-2 virus and the disease associated with this infection is COVID-19.

9. The method according to claim 1,
wherein the pharmaceutical composition containing Lip-APR is administered parenterally to a mammal.

10. The method according to claim 9,
wherein the parenteral administration is via intravenous injection, inhalation, or nasal administration.

11. A pharmaceutical composition comprising:
liposomal aprotinin (Lip-APR) in a therapeutically effective amount, wherein the Lip-APR is in the form of an aqueous dispersion or emulsion.

12. The pharmaceutical composition according to claim 11,
further comprising APR outside of the liposomes.

13. The pharmaceutical composition according to claim 11,
wherein the liposomes of the Lip-APR comprise phosphatidylcholine, cholesterol, distearoylphosphatidylethanolamine-(polyethylene glycol) and water for injection.

14. The pharmaceutical composition according to claim 11,
wherein the liposomes of the Lip-APR comprise phosphatidylcholine, cholesterol, and distearoylphosphatidylethanolamine-(polyethylene glycol).
